(19) United States
(12) Patent Application Publication
(10) Pub. No.: US 2018/0368748 A1
(43) Pub. Date: Dec. 27, 2018

(54) DEGREE-OF-INTEREST ESTIMATION DEVICE, DEGREE-OF-INTEREST ESTIMATION METHOD, PROGRAM, AND STORAGE MEDIUM

(71) Applicant: OMRON Corporation, Kyoto-shi, KYOTO (JP)

(72) Inventor: Haruka TANIGUCHI, Kyoto-shi (JP)

(73) Assignee: OMRON Corporation, Kyoto-shi, KYOTO (JP)

(21) Appl. No.: 15/753,203

(22) PCT Filed: Jan. 4, 2017
(86) PCT No.: PCT/JP2017/000048

§ 371 Date: Feb. 16, 2018

(30) Foreign Application Priority Data
Mar. 15, 2016 (JP) 2016-051481

(51) Int. Cl. A61B 5/16; A61B 5/024; A61B 5/103; G16H 50/30; G06Q 30/02

(52) U.S. Cl. CPC A61B 5/165; A61B 5/02405; A61B 5/02416; A61B 2560/0242; G16H 50/30; G06Q 30/0203; A61B 2503/12; A61B 5/1032

(57) ABSTRACT

A moving image of a crowd stimulated by an object is input. Based on the moving image, existence of each person constituting the crowd is recognized. A pulse of the person is obtained based on a luminance change of a skin of the person in the moving image. An attribute of the person in the moving image is recognized. The pulse of the person is corrected so as to eliminate a difference in pulse depending on the attribute. A statistical processing value of the pulse of the crowd is obtained by statistically processing the corrected pulse of the person. A numerical index corresponding to the statistical processing value of the pulse of the crowd is outputted as a degree-of-interest.

### TECHNICAL FIELD

The present invention relates to a degree-of-interest estimation device, more particularly to a degree-of-interest estimation device and a degree-of-interest estimation method for estimating a degree-of-interest of a crowd with respect to an object such as an event. The present invention also relates to a program causing a computer to perform the degree-of-interest estimation method. The present invention also relates to a computer-readable storage medium on which the program is recorded.

### BACKGROUND ART

Conventionally, for example, as disclosed in Patent Document 1 (Japanese Unexamined Patent Publication No. 2009-24775), an apparatus and a method for estimating a degree-of-interest of a human with respect to an object based on a human visual line speed and a skin potential have been known as this kind of degree-of-interest estimation device.

### PRIOR ART DOCUMENT

### Patent Document

Patent Document 1: Japanese Unexamined Patent Publication No. 2009-24775

### SUMMARY OF THE INVENTION

### Problems to be Solved by the Invention

In recent years, there is a need to estimate the degree-of-interest of the crowd with respect to the object such as the event.

However, so far as the inventor knows, there is no technique of estimating the degree-of-interest of the crowd with respect to the object. For example, in the degree-of-interest estimation device in Patent Document 1, because it is necessary to attach an electrode to a human skin, the degree-of-interest estimation device is not suitable for estimating the degree-of-interest of the crowd at once. When a statistical processing values (such as an average value) is obtained by directly performing statistical processing on a measurement result of each person, the obtained statistical processing value includes an individual difference variation in a normal state (when no stimulus is received from the object), and it is considered that accuracy of estimation is not good.

An object of the present invention is to provide a degree-of-interest estimation device and a degree-of-interest estimation method for being able to appropriately estimate the degree-of-interest of the crowd with respect to the object. Another object of the present invention is to provide a program causing a computer to perform the degree-of-interest estimation method. Another object of the present invention is to provide a computer-readable storage medium in which the program is recorded.

### Means for Solving the Problem

In order to solve the above-mentioned problem, according to one aspect of the present invention, a degree-of-interest estimation device that estimates a degree-of-interest of a crowd with respect to an object, the degree-of-interest estimation device includes: a moving image input unit configured to input a moving image in which the crowd stimulated by the object is photographed; a person recognizer configured to recognize existence of each person constituting the crowd based on the moving image, a pulse acquisition unit configured to obtain a pulse of the person based on a luminance change of a skin of the person in the moving image; an attribute recognizer configured to recognize an attribute of the person in the moving image; a first pulse correction unit configured to correct the pulse of the person so as to eliminate a difference in pulse depending on the attribute; a statistical processor configured to obtain a statistically processing value of the pulse of the crowd by statistically processing the corrected pulse of the person; and a degree-of-interest output unit configured to output a numerical index corresponding to the statistical processing value of the pulse of the crowd as the degree-of-interest.

As used herein, the term “object” means an object, such as an event, in which the crowd is interested.

The term “stimulated by the object” means that the crowd is stimulated through at least one of the five senses, that is, through at least one of sight, hearing, smell, taste, and touch.

The term “moving image input unit” means an input interface that inputs the moving image, for example.

The term “pulse” means a pulse rate per unit time, for example, a pulse rate per minute, namely, [beats/minute] (also expressed as [bpm]).

The term “statistical processing” means processing for obtaining an average value, a variance, and the like.

In the degree-of-interest estimation device of the present invention, the moving image input unit inputs the moving image in which the crowd stimulated by the object is photographed. The person recognizer recognizes the existence of the person constituting the crowd based on the moving image. Then, the pulse acquisition unit obtains the pulse of the person based on the luminance change of the skin of the person in the moving image. The attribute recognizer recognizes the attribute of the person in the moving image. Then, the first pulse correction unit corrects the pulse of the person so as to eliminate the difference in pulse depending on the attribute. Consequently, the corrected pulse of the person expresses a difference in pulse when the pulse is stimulated by the object with respect to the pulse of the person in a normal state (when the pulse is not stimulated by the object) while a variation due to the attribute is eliminated. Then, the statistical processor statistically processes the corrected pulse of the person, and obtains the statistical processing value of the pulse of the crowd. As a result, the statistical processing value of the pulse of the crowd expresses the difference in pulse when the pulse is stimulated by the object with respect to the pulse of the crowd in the normal state while the variation due to the attribute is eliminated. Then, the degree-of-interest output unit outputs the numerical index corresponding to the statistical processing value of the pulse of the crowd as the degree-of-interest. Thus, in the degree-of-interest estimation device, the degree-of-interest of the crowd with respect to the object can appropriately be estimated.

For example, the statistical processor obtains a statistical processing value of a pulse of a first crowd and a statistical processing value of a pulse of a second crowd at a certain time point, and the degree-of-interest output unit outputs a numerical index corresponding to a difference between the statistical processing value of the pulse of the first crowd and the statistical processing value of the pulse of the second crowd as the degree-of-interest. Alternatively, the statistical processor obtains a statistical processing value of a pulse at a first time point and a statistical processing value of a pulse at a second time point for a certain crowd, and the degree-of-interest output unit outputs a numerical index corresponding to a difference between the statistical processing value of the pulse at the first time point and the statistical processing value of the pulse at the second time point as the degree-of-interest.

The degree-of-interest estimation device of an embodiment includes: an environment information input unit configured to input environment information indicating an environment surrounding the photographed crowd; a second pulse correction unit configured to correct the statistical processing value of the pulse of the crowd so as to eliminate a difference in pulse depending on the environment based on the environmental information obtained by the environment information input unit.

As used herein, for example, the term “environment information” means a temperature surround the crowd.

In the degree-of-interest estimation device of an embodiment, the environment information input unit inputs the environment information indicating the environment surrounding the photographed crowd. The second pulse correction unit corrects the statistical processing value of the pulse of the crowd so as to eliminate the difference in pulse depending on the environment based on the environment information obtained by the environment information input unit. The corrected statistical processing value becomes one in which the variation depending on the environment is eliminated. Then, the degree-of-interest output unit outputs the numerical index corresponding to the statistical processing value of the pulse of the crowd as the degree-of-interest. Thus, in the degree-of-interest estimation device, the degree-of-interest of the crowd with respect to the object can further appropriately be estimated.

In the degree-of-interest estimation device of an embodiment, the attribute of the person is at least one of age and sexuality.

In the degree-of-interest estimation device according to this embodiment, the pulse of the person corrected by the first pulse correction unit is in a state in which the variation due to at least one of the age and the sexuality is eliminated. Thus, the degree-of-interest of the crowd with respect to the object can appropriately be estimated.

In the degree-of-interest estimation device of an embodiment, the first pulse correction unit performs a correction by multiplying the pulse of the person obtained by the pulse acquisition unit by a predetermined pulse correction coefficient by age and a predetermined pulse correction coefficient by sexuality according to at least one of the age and the sexuality, which are recognized by the attribute recognizer.

In the degree-of-interest estimation device of an embodiment, the first pulse correction unit performs the correction by multiplying the pulse of the person obtained by the pulse acquisition unit by a predetermined pulse correction coefficient by age and a predetermined pulse correction coefficient by sexuality according to at least one of the age and the sexuality, which are recognized by the attribute recognizer. This enables the pulse to be easily corrected.

The degree-of-interest estimation device of an embodiment further includes an imaging unit configured to acquire the moving image by photographing the crowd stimulated by the object.

In the degree-of-interest estimation device of an embodiment, the imaging unit acquires the moving image by photographing the crowd stimulated by the object.

According to another aspect of the present invention, a degree-of-interest estimation method for estimating a degree-of-interest of a crowd with respect to an object, the degree-of-interest estimation method includes the steps of: inputting a moving image in which the crowd stimulated by the object is photographed; recognizing existence of each person constituting the crowd based on the moving image; obtaining the pulse of the person based on a luminance change of a skin of the person in the moving image; recognizing an attribute of the person in the moving image; correcting the pulse of the person so as to eliminate a difference in pulse depending on the attribute; obtaining a statistical processing value of the pulse of the crowd by statistically processing the corrected pulse of the person; and outputting a numerical index corresponding to the statistical processing value of the pulse of the crowd as the degree-of-interest.

In the degree-of-interest estimation method of the present invention, through the processing of “correcting the pulse of the person so as to eliminate the difference in pulse depending on the attribute”, the corrected pulse of the person expresses a change in pulse when the pulse is stimulated by the object with respect to the pulse of the person in a normal state (when the pulse is not stimulated by the object) while a variation due to the attribute is eliminated. As a result, the statistical processing value of the pulse of the crowd obtained through the processing of “obtaining the statistical processing value of the pulses of the crowd by statistically processing the corrected pulses of the person” expresses the change in pulse when the pulse is stimulated by the object with respect to the pulse of the person in the normal state while the variation due to the attribute is eliminated. Then, the numerical index corresponding to the statistical processing value of the pulse of the crowd is output as the degree-of-interest. Thus, in the degree-of-interest estimation method, the degree-of-interest of the crowd with respect to the object can appropriately be estimated.

Either the processing of “obtaining the pulse of the person based on the luminance change of the skin of the person in the moving image” or the processing of “recognizing the attribute of the person in the moving image” may be performed in advance, or both the pieces of processing may be performed in parallel.

In still another aspect of the present invention, a program causes a computer to perform the degree-of-interest estimation method of the invention.

In the program of the present invention, the computer can be caused to perform the degree-of-interest estimation method of the invention.

In yet another aspect of the present invention, a storage medium of the invention is a computer-readable storage medium in which the program of the invention is recorded.

When the program recorded in the storage medium of the present invention is installed in a computer, the computer can be caused to perform the degree-of-interest estimation method of the invention.

### Effect of the Invention

As is clear from the above, in the degree-of-interest estimation device and degree-of-interest estimation method of the present invention, the degree-of-interest of the crowd with respect to the object can appropriately be estimated. In the program of the present invention, the computer can be caused to perform the degree-of-interest estimation method of the invention. In the program recorded in the storage medium of the present invention, the computer can be caused to perform the degree-of-interest estimation method of the invention.

### BRIEF DESCRIPTION OF THE DRAWINGS

FIG. 1 is a diagram illustrating a block configuration of a degree-of-interest estimation device according to an embodiment of the present invention.

FIG. 2 is an overall processing flowchart of a degree-of-interest estimation method performed by the degree-of-interest estimation device.

FIG. 3 is a diagram illustrating an example of step S9 of comparing a pulse average value of a crowd in FIG. 2.

FIG. 4 is a graph illustrating a pulse average value B11 of a first crowd B1 and a pulse average value B21 of a second crowd B2 at a certain time t1.

FIG. 5 is a diagram illustrating another example of step S9 of comparing the pulse average value of the crowd in FIG. 2.

FIG. 6 is a graph illustrating time lapses of pulse average values of crowds C1 and C2.

FIG. 7 is a flowchart of a modification of steps S9 and S10 in FIG. 2.

FIG. 8(A) is a graph illustrating pulse distributions of crowds D1 and D2. FIG. 8(B) is a graph illustrating pulse distributions of the crowd D1 and D2′.

FIG. 9 is a flowchart of another modification of steps S9 and S10 in FIG. 2.

### MODE FOR CARRYING OUT THE INVENTION

Hereinafter, an embodiment of the present invention will be described in detail with reference to the drawings.

(Schematic Configuration of Apparatus)

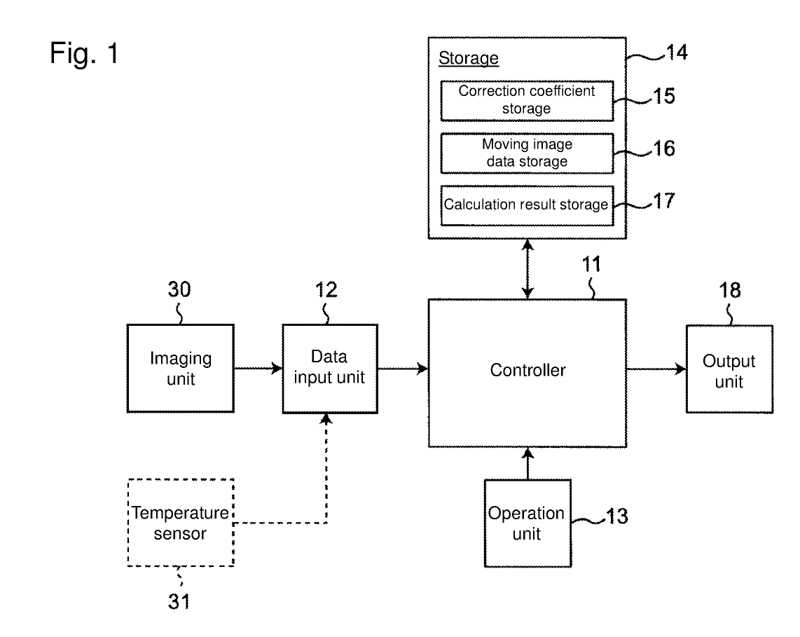

FIG. 1 illustrates a block configuration of a degree-of-interest estimation device according to an embodiment of the present invention.

The degree-of-interest estimation device includes a controller 11, a data input unit 12, an operation unit 13, a storage 14, and an output unit 18. An imaging unit 30 is connected to the data input unit 12.

The imaging unit 30 photographs a crowd that is stimulated by an object, and acquires a moving image. The imaging unit 30 is a commercially available photographing camera. However, the imaging unit 30 is not limited thereto.

The controller 11 includes a central processing unit (CPU) that is operated by software, and performs various pieces of processing (to be described later).

The data input unit 12 is constructed with a known input interface, and sequentially inputs moving image data acquired by the imaging unit 30 in real time.

The operation unit 13 includes known keyboard and mouse, and serves to input a command and various pieces of information from a user. For example, the command includes a command instructing start of the processing and a command instructing recording of a calculation result. For example, the input information includes time (year, month, day, and time) the moving image was photographed and information identifying a plurality of pieces of moving image data input by the data input unit 12.

The storage 14 is constructed with a hard disk drive or an electrically rewritable nonvolatile memory (EEPROM) that can non-transiently store data, and includes a correction coefficient storage 15, a moving image data storage 16, and a calculation result storage 17.

The correction coefficient storage 15 stores a pulse correction coefficient for correcting a pulse of each person so as to eliminate a difference in pulse depending on an attribute of each person constituting the crowd. In the embodiment, “pulse correction coefficient table by age” in Table 1 and “pulse correction coefficient table by sexuality” in Table 2 are stored in the correction coefficient storage 15. These pulse correction coefficients are set so as to eliminate a variation in pulse average value based on general knowledge such as the fact that a pulse average value of a child during a normal time (when the child is not stimulated by the object) tends to be higher than a pulse average value of an adult during the normal time, the fact that a pulse average value of a female tends to be higher than a pulse average value of a male, and the fact that elderly person have a low maximum pulse rate. Specifically, a pulse correction coefficient α by age in Table 1 corresponds to a factor equalizing the pulse average value of persons except for 19- to 59-year-old adults (0- to 6-year-old infants, 7- to 12-year-old children and elementary school students, 13- to 18-year-old junior high school students and high school students, and elderly people of 60 and above) to the pulse average value of adults based on the pulse average value of adults. In addition, a pulse correction coefficient β by sexuality in Table 2 corresponds to a factor equalizing the pulse average value of the female to the pulse average value of the male based on the pulse average value of the male.

TABLE 1
Pulse correction coefficient table by age

| | | Pulse | | |
|---|---|---|---|---|
| | | rate | Pulse | Pulse |
| | | range | average | correction |
| | Age [years] | [bpm] | value [bpm] | coefficient α |
| Infants | 0-6 | 100-140 | 120 | 0.583333333 |
| Children and | 7-12 | 70-110 | 90 | 0.777777778 |
| elementary school | | | | |
| students | | | | |
| Junior high school | 13-18 | 60-100 | 80 | 0.875 |
| students and high | | | | |
| school students | | | | |
| Adults | 19-59 | 50-90 | 70 | 1 |
| Elderly people | 60 and above | 60-80 | 70 | 1 |

TABLE 2
Pulse correction coefficient table by sexuality

| | Pulse average value | Pulse correction |
|---|---|---|
| Sexuality | [bpm] | coefficient β |
| Male | 65 | 1 |
| Female | 70 | 0.928571429 |

The moving image data storage 16 in FIG. 1 stores the moving image data input through the data input unit 12 while correlating each moving image to an identification number of the moving image.

For each moving image, the calculation result storage 17 stores a numerical index indicating the degree-of-interest of the crowd with respect to the object obtained by the processing (to be described later) while correlating the numerical index to the identification number of the moving image.

The output unit 18 is constructed with a liquid crystal display (LCD), and displays various pieces of information such as a calculation result of the controller 11. The output unit 18 may include a printer (driver) and print out the calculation result on paper.

A temperature sensor 31 is any optional additional element that detects a temperature [° C.] as environmental information indicating an environment surrounding the photographed crowd. The data input unit 12 acts as an environment information input unit, thereby inputting the temperature [° C.] detected to the controller 11.

(Degree-of-Interest Estimation Method)

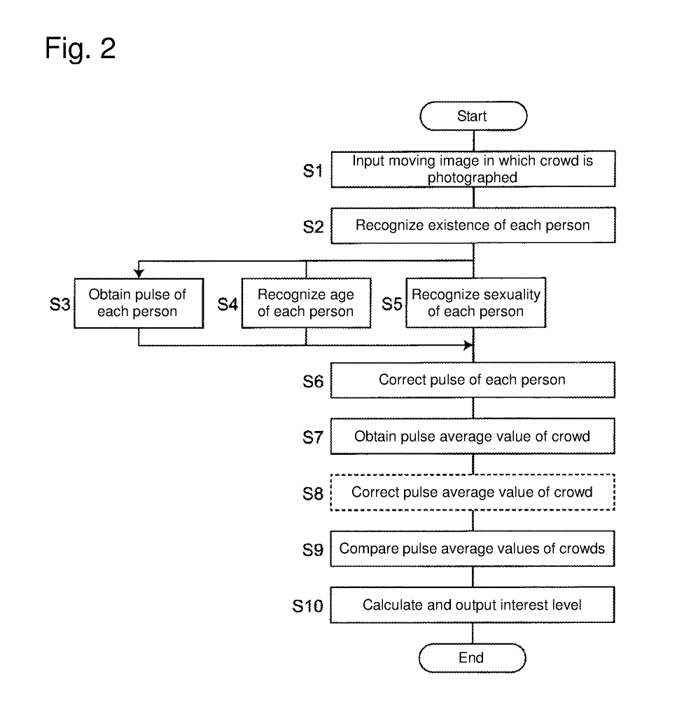

The degree-of-interest estimation device is operated according to an overall processing flowchart in FIG. 2 under the control of the controller 11.

(1) Input of Moving Image

First, as illustrated in step S1 of FIG. 2, the controller 11 inputs data of the moving image photographed by the imaging unit 30 through the data input unit 12.

It is assumed that the crowd stimulated by the object is photographed in the moving image. In the embodiment, it is assumed that a crowd watching an event such as an exhibition and a lecture is photographed.

The moving image data photographed by the imaging unit 30 is sequentially input in real time through the data input unit 12, and the moving image data is stored in the moving image data storage 16 under the control of the controller 11 while correlated to the identification number of the moving image.

In the embodiment, the moving image data is sequentially photographed by the imaging unit 30. However, the present invention is not limited thereto. The data input unit 12 may sequentially or substantially simultaneously receive and input the moving image data, which is previously acquired outside the degree-of-interest estimation device, through a network (not shown) such as the Internet.

(2) Recognition of Existence of Each Person

Then, the controller 11 acts as a person recognizer, and recognizes existence of each person constituting the crowd based on the moving image as illustrated in step S2 of FIG. 2. The existence of each person is recognized in each image constituting the moving image by a known technique disclosed in, for example, Paul Viola et al. “Rapid Object Detection using a Boosted Cascade of Simple Features” Computer Vision and Pattern Recognition, 2001. CVPR 2001. Proceedings of the 2001 IEEE Computer Society Conference on 2001, P. 1-511-1-518 vol. 1.

In the embodiment, it is assumed that persons Nos. 1 to 6 are recognized for a certain crowd as indicated in the leftmost column in Table 3.

(3) Pulse Acquisition

Then, the controller 11 acts as a pulse acquisition unit, and obtains the pulse of each person based on a luminance change of a skin of each person in the moving image as illustrated in step S3 of FIG. 2. Specifically, the pulse of each person is obtained based on the luminance change of the green component of the skin of each person by a known technique disclosed in, for example, Xiaobai Li et al. “Remote Heart Rate Measurement From Face Videos Under Realistic Situations” Computer Vision and Pattern Recognition (CVPR), 2014 IEEE Conference on 23-28 Jun. 2014, P. 4264-4271.

In the embodiment, it is assumed that the pulses of persons Nos. 1 to 6 at a certain time point are 110 [bpm], 90 [bpm], 75 [bpm], 63 [bpm], 75 [bpm], and 70 [bpm] as indicated in a field of “original pulse” in Table 3.

(4) Recognition of Age of Each Person

Then, the controller 11 acts as an attribute recognizer, and recognizes age of each person in the moving image as one of attributes by a known technique disclosed in, for example, Japanese Unexamined Patent Publication No. 2005-148880 as illustrated in step S4 of FIG. 2.

In the embodiment, as indicated in a field of “age” in Table 3, it is assumed that the ages of the persons Nos. 1 to 6 are 5 [years old], 10 [years old], 15 [years old], 20 [years old], 30 [years old], and 70 [years old]. This means that the person No. 1 belongs to a group of (0- to 6-year-old) infants in Table 1 listed above, the person No. 2 belongs to a group of (7- to 12-year-old) children and elementary school students, the person No. 3 belongs to a group of (13- to 18-year-old) junior high school students and high school students, the persons Nos. 4 and 5 belong to a group of (19- to 59-year-old) adults, and the person No. 6 belongs to a group of elderly people (of 60 and above).

(5) Recognition of Sexuality of Each Person

Then, the controller 11 acts as the attribute recognizer, and recognizes the age of each person in the moving image as another one of the attributes by a known technique disclosed in, for example, Japanese Unexamined Patent Publication No. 2010-33474 as illustrated in step S5 of FIG. 2.

In the embodiment, as indicated in a field of “Sexuality” in Table 3, it is assumed that the sexualities of the persons Nos. 1 to 6 are male, female, female, male, female, and male.

Any one of the pieces of processing (3) to (5) may be performed first, or performed in parallel with each other.

(6) Correction of Pulse of Each Person

Then, the controller 11 acts as a first pulse correction unit, and corrects the pulse of each person obtained through the processing (3) as illustrated in step S6 of FIG. 2. Specifically, the pulse of each person is corrected using the following equation EQ1 so as to eliminate a difference in pulse depending on the age and sexuality as the attribute.

That is, let n₀ [bpm] be the pulse (referred to as the “original pulse”) of each person obtained through the processing (3), and let the corrected pulse of each person be n₁ [bpm], using

n₁=n₀×α×β (EQ1),

the pulse of each person is corrected. Where a indicates the pulse correction coefficient by age in Table 1. β indicates the pulse correction coefficient by sexuality in Table 2.

In the embodiment, for the person No. 1, because the person No. 1 belongs to the group of (0- to 6-year-old) infants in Table 1, the pulse correction coefficient α by age is α=0.583333333. For the person No. 2, because the person No. 2 belongs to the group of (7- to 12-year-old) children and elementary school students, the pulse correction coefficient α by age is α=0.777777778. For the person No. 3, because the person No. 3 belongs to the group of (13- to 18-year-old) junior high school students and high school students, the pulse correction coefficient α by age is α=0.875. For the persons Nos. 4 and 5, because the persons Nos. 4 and 5 belong to the group of (19- to 59-year-old) adults, the pulse correction coefficient α by age is α=1. For the person No. 6, because the person No. 6 belongs to the group of elderly people (of 60 and above), the pulse correction coefficient α by age is α=1.

In the embodiment, the sexuality of each of Nos. 1 to 6 is male, female, female, male, female, and male. Consequently, for the persons Nos. 1, 4, and 6, the pulse correction coefficient β by sexuality is β=1. For the persons Nos. 2, 3, and 5, the pulse correction coefficient β by sexuality is β=0.928571429.

Thus, when the correction is performed according to the equation EQ1, the corrected pulse n₁ of each person becomes n₁=64.16666663 for the person No. 1 as indicated in Table 3. For the person No. 2, n₁=65.00000005. For the person No. 3, n₁=60.93750003. For the person No. 4, n₁=63. For the person No. 5, n₁=69.64285717. For the person No. 6, n₁=70.

TABLE 3
Example of pulse correction by attribute

| | | | Original | Correction | Correction | Corrected |
|---|---|---|---|---|---|---|
| | Attributes | | pulse | coefficient | coefficient | pulse |
| N0. | Age [years] | Sexuality | [bpm] | α by age | β by sexuality | [bpm] |
| 1 | 5 | Male | 110 | 0.583333333 | 1 | 64.16666663 |
| 2 | 10 | Female | 90 | 0.777777778 | 0.928571429 | 65.00000005 |
| 3 | 15 | Female | 75 | 0.875 | 0.928571429 | 60.93750003 |
| 4 | 20 | Male | 63 | 1 | 1 | 63 |
| 5 | 30 | Female | 75 | 1 | 0.928571429 | 69.64285717 |
| 6 | 70 | Male | 70 | 1 | 1 | 70 |

As described above, the pulse correction coefficient α by age in Table 1 corresponds to the factor equalizing the pulse average value of persons except for 19- to 59-year-old adults (0- to 6-year-old infants, 7- to 12-year-old children and elementary school students, 13- to 18-year-old junior high school students and high school students, and elderly people of 60 and above) to the pulse average value of adults based on the pulse average value of adults. In addition, a pulse correction coefficient β by sexuality in Table 2 corresponds to a factor equalizing the pulse average value of the female to the pulse average value of the male based on the pulse average value of the male. Consequently, by multiplying the pulse correction coefficient α by age and the pulse correction coefficient β by sexuality with respect to the original pulse no according to the equation EQ1, the corrected pulse n₁ of each person expresses a difference in pulse when each person is stimulated by the object with respect to the pulse of each person during the normal time (when each person is not stimulated by the object) while the variation due to the age and sexuality is eliminated. This enables the pulse to be easily corrected.

For the original pulse of each person, the correction may be performed for not both the age and the sexuality, but either the age or the sexuality.

The pulse of each person may be corrected by not multiplying the correction coefficient α by age and the correction coefficient β by sexuality, but adding or subtracting a predetermined correction pulse rate.

(7) Calculation of Pulse Average Value of Crowd

Then, the controller 11 acts as a statistical processor, statistically processes the corrected pulse n₁ of each person obtained through the processing (6) as illustrated in step S7 of FIG. 2, and obtains a statistical processing value of the pulse of the crowd. In the embodiment, an average value is obtained as the statistical processing value. The obtained average value is referred to as “a pulse average value of the crowd” (denoted by a symbol N₁, and the unit is [bpm]).

In the embodiment, the pulse average value N₁ of the crowd including the persons Nos. 1 to 6 at a certain time point is obtained as follows.

N₁=(64.16666663+65.00000005+60.93750003+63+69.64285717+70)/6=65.45783731 [bpm]

Other statistical processing values such as a median and a variance may be adopted as the statistical processing value of the pulse of the crowd instead of the average value.

(8) Correction of Pulse Average Value of Crowd

Then, the controller 11 acts as a second pulse correction unit, and corrects the pulse average value N₁ of the crowd obtained through the processing (7) as illustrated in step S8 of FIG. 2. Step S8 is any optional additional step, and the frame of step S8 is indicated by a broken line in order to indicate the optional additional step.

Specifically, the controller 11 receives the temperature [° C.] through the data input unit 12 as the environmental information indicating the environment surrounding the crowd detected by the temperature sensor 31. Then, the controller 11 acts as the second pulse correction unit, and corrects the pulse average value N₁ of the crowd so as to eliminate the difference in pulse depending on the above temperature [° C.] by a known technique disclosed in, for example, Japanese Unexamined Patent Publication No. H8-080287 (correcting an influence of a temperature change of the pulse rate, and displaying the pulse rate under a certain condition).

An oxygen concentration may be used instead of or in addition to the temperature as environmental information indicating the environment surrounding the crowd. In this case, the pulse average value N₁ of the crowd is corrected so as to eliminate the difference in pulse depending on the oxygen concentration.

Hereinafter, it is assumed that the pulse average value of the crowd corrected through the processing in step S8 is denoted by a symbol N₂. When step S8 is omitted, the corrected pulse average value N₂ of the crowd is equal to the uncorrected pulse average value N₁ of the crowd.

(9) Comparison of Pulse Average Value of Crowd

Then, as illustrated in step S9 of FIG. 2, the controller 11 compares the pulse average value N₂ of the crowd obtained through the processing (8).

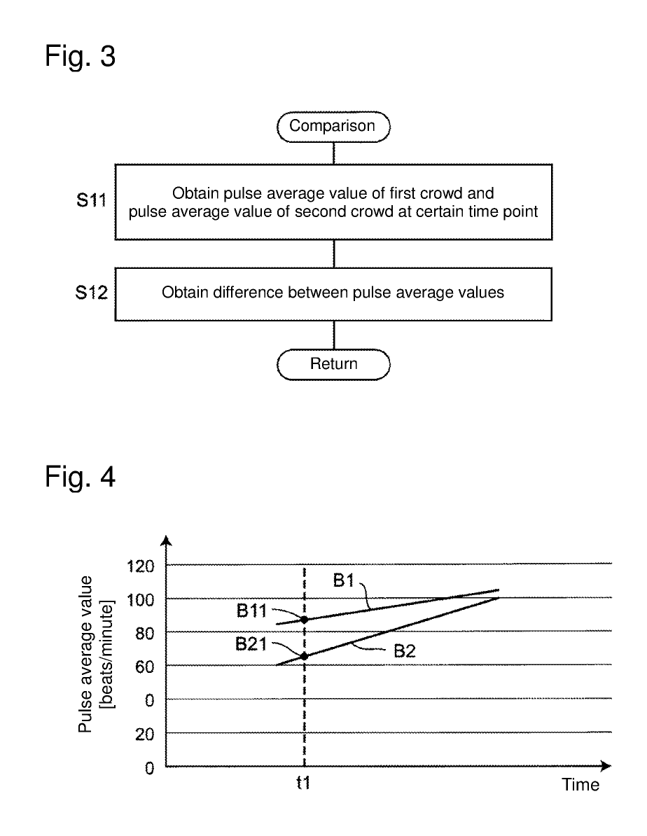

As an example, as illustrated in FIG. 4, it is assumed that the pulse average value N₂ of a first crowd B1 and the pulse average value N₂ of a second crowd B2 are B11 and B21 at a certain time t1, respectively. In this case, as illustrated in step S11 of FIG. 3, the controller 11 obtains the pulse average value B11 of the first crowd and the pulse average value B21 of the second crowd at the time t1. Then, a difference (denoted by a symbol ΔN) between the pulse average values is obtained as illustrated in step S12. In this case, the difference ΔN between the pulse average values is calculated as follows.

ΔN=|B11−B21|

A direction of the subtraction is set such that a symbol of the difference ΔN becomes positive.

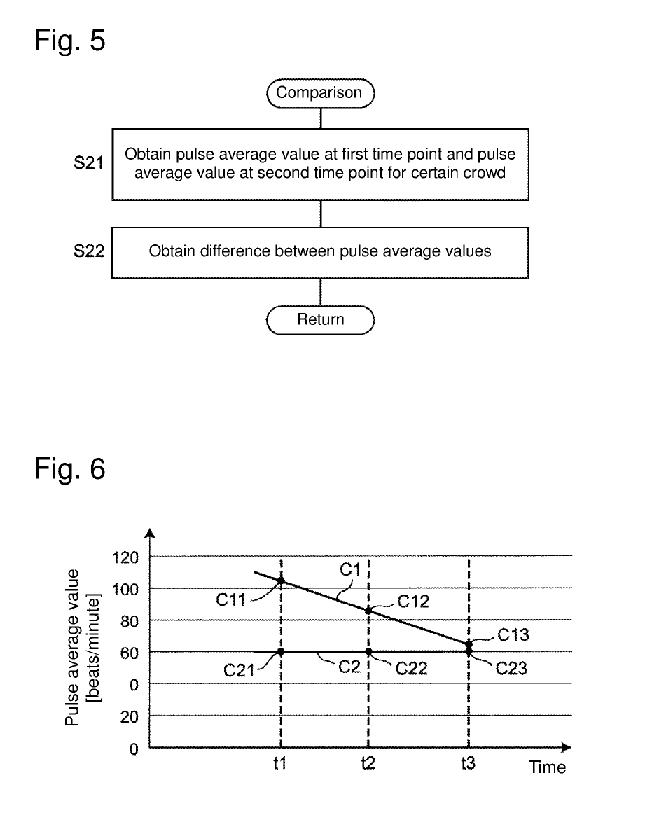

As another example, as illustrated in FIG. 6, it is assumed that the pulse average value N₂ at the first time point t1 and the pulse average value N₂ at a second time point t2 are C11 and C12 for a certain crowd C1, respectively. In this case, as illustrated in step S21 of FIG. 5, the controller 11 obtains the pulse average value C11 at the first time point t1 and the pulse average value C12 at the second time point t2 for the crowd C1. Then, the difference ΔN between the pulse average values is obtained as illustrated in step S22. In this case, the difference ΔN between the pulse average values is calculated as follows.

ΔN=|C11−C12|

(10) Calculation and Output of Degree-of-Interest

Then, as illustrated in step S10 of FIG. 2, the controller 11 and the output unit 18 act as a degree-of-interest output unit, and output the numerical index according to the difference ΔN between the pulse average values obtained through the processing (9) as the degree-of-interest (this is denoted by a symbol X) of the crowd with respect to the object.

In the embodiment, as indicated in Table 4, a correspondence table in which the difference ΔN between the pulse average values and the degree-of-interest X are correlated to each other is prepared in advance (for example, the correspondence table is stored in the storage 14 of FIG. 1). The correspondence table indicates that the greater the difference ΔN between the pulse average values, the higher the degree-of-interest X in stages. Specifically, when the difference ΔN is less than 5, the degree-of-interest X=1. When the difference ΔN is greater than or equal to 5 and less than 15, the degree-of-interest X=2. When the difference ΔN is greater than or equal to 15 and less than 25, the degree-of-interest X=3. When the difference ΔN is greater than or equal to 25 and less than 35, the degree-of-interest X=4. When the difference ΔN is greater than or equal to 35, the degree-of-interest X=5.

TABLE 4
Correspondence table of difference between pulse average values
with degree-of-interest

| Difference ΔN between pulse average values | |
|---|---|
| [bpm] | Interest level X |
| Greater than or equal to 35 | 5 |
| Greater than or equal to 25 and less than 35 | 4 |
| Greater than or equal to 15 and less than 25 | 3 |
| Greater than or equal to 5 and less than 15 | 2 |
| Less than 5 | 1 |

For example, in the example illustrated in FIG. 4, the pulse average value N₂ of the first crowd B1 and the pulse average value N₂ of the second crowd B2 are B11 and B21 at the certain time t1, respectively. Referring to a vertical axis in FIG. 4, the difference ΔN between the pulse average values is given as follows.

ΔN=B11−B21≈20 [bpm]

At the time t1, the degree-of-interest X of the first crowd B1 is estimated to be higher by 3 than the degree-of-interest of the second crowd B2 according to the correspondence table in Table 4.

In the example illustrated in FIG. 6, the pulse average value N₂ at the first time point t1 and the pulse average value N₂ at the second time point t2 are C11 and C12 for the certain crowd C1, respectively. Referring to the vertical axis of FIG. 6, the difference ΔN between the pulse average values is given as follows.

ΔN=C11−C12≈20 [bpm]

At this point, for the crowd C1, the degree-of-interest X at the first time point t1 is estimated to be higher by 3 than the degree-of-interest at the second time point t2 according to the correspondence table in Table 4.

Thus, in the degree-of-interest estimation device, the degree-of-interest of the crowd with respect to the object can appropriately be estimated.

(First Modification)

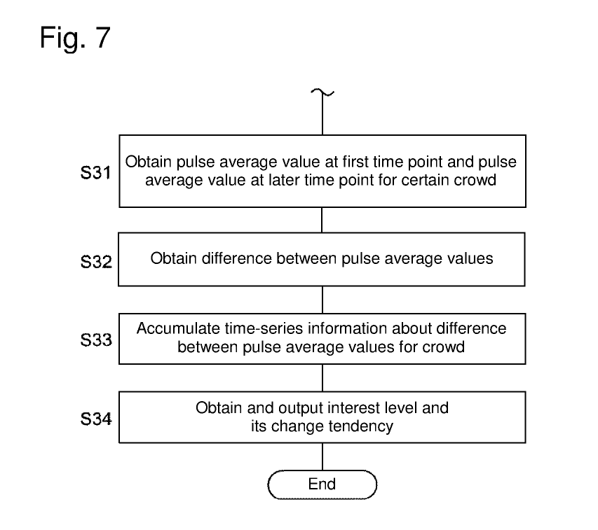

FIG. 7 illustrates a flowchart of a modification of steps S9 and S10 in FIG. 2. In the flowchart of the first modification, as illustrated in step S31 of FIG. 7, the controller 11 obtains the pulse average value at the first time point and the pulse average value at the later time point for the certain crowd. Then, the difference ΔN between the pulse average values is obtained as illustrated in step S32. For the crowd, time-series information about the difference ΔN between the pulse average values is accumulated as illustrated in step S33. Then, as illustrated in step S34, the degree-of-interest X and its change tendency for the crowd are obtained and output.

In the example illustrated in FIG. 6, for the certain crowd C1, the pulse average value N₂ at the first time point t1, the pulse average value N₂ at the second time point t2, and the pulse average value N₂ at a third time point t3 are C11, C12, C13, respectively. As described above, between the first time point t1 and the second time point t2, the difference ΔN between the pulse average values is given as follows.

ΔN=C11−C12≈20 [bpm]

At this point, for the crowd C1, the degree-of-interest X at the first time point t1 is estimated to be higher by 3 than the degree-of-interest at the second time point t2 according to the correspondence table in Table 4. Conversely, it can be said that the degree-of-interest X at the second time point t2 is lower by 3 than the degree-of-interest at the first time point t1. Then, between the second time point t2 and the third time point t3, the difference ΔN between the pulse average values is given as follows.

ΔN=C12−C13≈20 [bpm]

At this point, according to the correspondence table in Table 4, for the crowd C1, the degree-of-interest X at the second time point t2 is estimated to be higher by 3 than the degree-of-interest at the third time point t3. Conversely, it can be said that the degree-of-interest X at the third time point t3 is lower by 3 than the degree-of-interest at the second time point t2. As a result, it is understood that the degree-of-interest for the crowd C1 tends to decrease (i.e., the crowd C1 gets bored) with the lapse of time.

For another crowd C2 in FIG. 6, the pulse average value N₂ at the first time point t1, the pulse average value N₂ at the second time point t2, and the pulse average value N₂ at the third time point t3 are C21, C22, C23, respectively, and remain low and are hardly changed (C21=C22=C23=60 [bpm]). In this case, for the crowd C2, it is understood that there is no degree-of-interest.

In the first modification, the controller 11 outputs the degree-of-interest X and its change tendency obtained in this way. Thus, the degree-of-interest of the crowd with respect to the object can further appropriately be estimated.

(Second Modification)

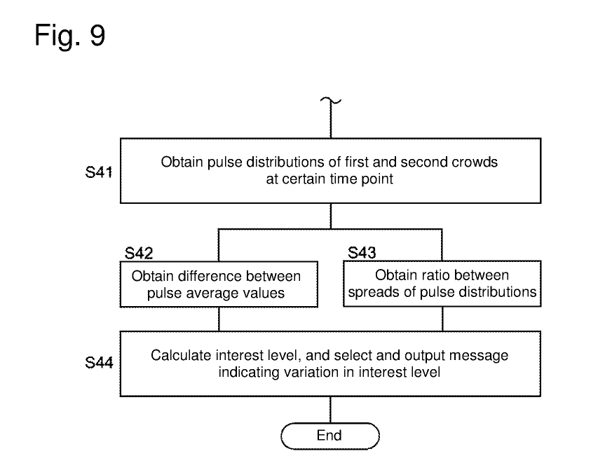

FIG. 9 illustrates a flowchart of another modification of steps S9 and S10 in FIG. 2. In step S7 of FIG. 2, not only the pulse average value but also a pulse distribution are obtained as the statistical processing value of the pulse of the crowd.

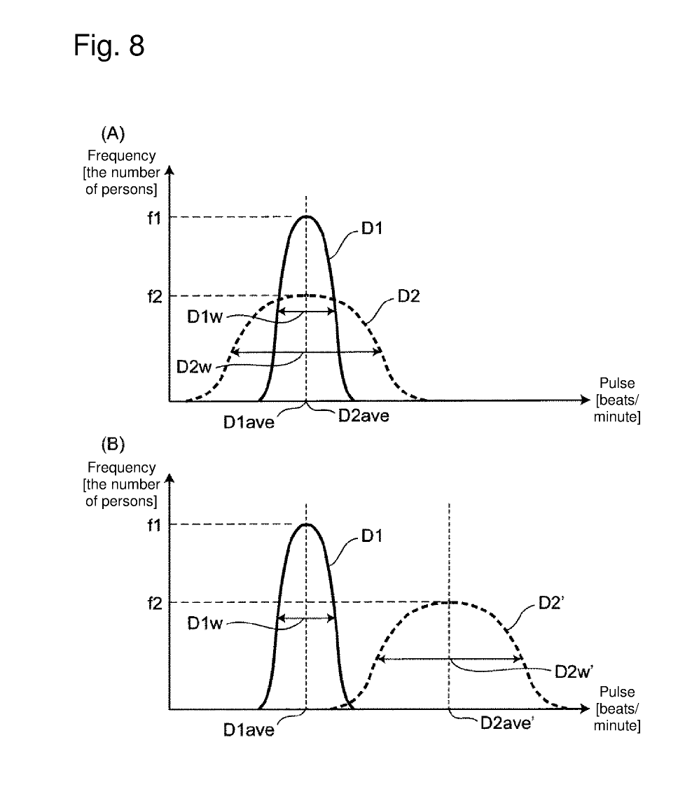

As used herein, the pulse distribution means a distribution when a horizontal axis indicates the pulse [beats/minute] of each person while a vertical axis indicates a frequency [the number of persons] for the crowds D1 and D2 in FIG. 8(A). In the second modification, it is assumed that sizes (the number of persons) of the crowds D1 and D2 are sufficiently large, and that each pulse distribution is regarded as a normal distribution. In this case, a shape of each pulse distribution is specified by pulse average values D1ave and D2ave and a normalized spread (half value width/frequency) of the pulse distribution. For the crowds D1 and D2 in FIG. 8(A), the pulse average values D1ave and D2ave are equal to each other, namely, D1ave=D2ave. The normalized spreads (D1w/f1) and (D2w/f2) of the pulse distributions for the crowds D1 and D2 are different from each other, namely, (D1w/f1)<(D2w/f2). In such cases, the variation in degree-of-interest of each person constituting the crowd is not estimated only by obtaining the degree-of-interest X based on the pulse average values D1ave and D2ave.

Consequently, in the flowchart of the second modification, the controller 11 obtains the pulse distribution of the first crowd and the pulse distribution of the second crowd at a certain time point as illustrated in step S41 of FIG. 9. Then, the difference ΔN between the pulse average values is obtained as illustrated in step S42. At the same time, a ratio between the normalized spreads of the pulse distributions is obtained as illustrated in step S43. Then, as illustrated in step S44, the degree-of-interest X is calculated, and a message indicating the variation in degree-of-interest is selected and outputted.

For example, with respect to the crowd D2 illustrated in FIG. 8(A), although there is no difference in degree-of-interest X for the crowd D1, a message indicating that “the degree-of-interest varies largely” is outputted.

For a crowd D2′ illustrated in FIG. 8(B), a pulse average value D2ave′ is larger than that of the crowd D2 in FIG. 8(A) and a normalized spread of a pulse distribution (D2w′/f2′) is equal to that of the crowd D2. In this case, with respect to the crowd D2′, the degree-of-interest X is indicated to the crowd D1 and a message indicating “the variation in the degree-of-interest is large” is outputted.

Various messages such as “the variation in the degree-of-interest is large” and “the variation in the degree-of-interest is small” are prepared in advance (stored in the storage 14 in FIG. 1), and the controller 11 desirably selects the messages according to the ratio of the normalized spreads of the pulse distributions.

In the embodiment, the pulse correction coefficient α by age and the pulse correction coefficient β by sexuality are independently set so as to eliminate the difference in pulse depending on the age and the sexuality as the attribute. However, the present invention is not limited thereto Alternatively, for example, the age may be taken in a row direction, the sexuality may be taken in a column direction, and the pulse correction coefficient may be set as an element of a matrix in which the age and the sexuality are combined. In such cases, for example, a specific tendency in which the age and the sexuality are combined such that the pulse of the female of 50 and above rises easily can be corrected. That is, for the female of 50 and above, the difference in pulse depending on the age and the sexuality as the attribute can be eliminated when the correction is performed so as to reduce a width of pulse increase.

Further, in the embodiment, the moving image is photographed and acquired. However, the present invention is not limited thereto. The photographed moving image may be input and acquired through a network such as the Internet and a local area network.

The degree-of-interest estimation method can be recorded in a storage medium, such as a compact disk (CD), a digital versatile disk (DVD), and a flash memory, in which data can non-transiently be stored, as application software (computer program). The application software recorded in the storage medium is installed in a substantial computer device such as a personal computer, a personal digital assistant (PDA), and a smartphone, which allows the computer device to perform the degree-of-interest estimation method.

The embodiments are illustrative, and various modifications can be made without departing from the scope of the present invention. Each of the embodiments can be established independently, but it is also possible to combine the embodiments. In addition, various features in different embodiments can also be established independently, and a combination of features in different embodiments is also possible.

### DESCRIPTION OF SYMBOLS

11 controller
12 data input unit
13 operation unit
14 storage
18 output unit
30 imaging unit
31 temperature sensor

1. A degree-of-interest estimation device for estimating a degree-of-interest of a crowd with respect to an object, the degree-of-interest estimation device comprising:
a moving image input unit configured to input a moving image in which the crowd stimulated by the object is photographed;
a person recognizer configured to recognize existence of each person constituting the crowd based on the moving image;
a pulse acquisition unit configured to obtain a pulse of the person based on a luminance change of a skin of the person in the moving image;
an attribute recognizer configured to recognize an attribute of the person in the moving image;
a first pulse correction unit configured to correct the pulse of the person so as to eliminate a difference in pulse depending on the attribute;
a statistical processor configured to obtain a statistical processing value of the pulse of the crowd by statistically processing the corrected pulse of the person; and
a degree-of-interest output unit configured to output a numerical index corresponding to the statistical processing value of the pulse of the crowd as the degree-of-interest.

2. The degree-of-interest estimation device according to claim 1, further comprising:
an environment information input unit configured to input environment information indicating an environment surrounding the photographed crowd; and
a second pulse correction unit configured to correct the statistical processing value of the pulse of the crowd so as to eliminate a difference in pulse depending on the environment based on the environmental information obtained by the environment information input unit.

3. The degree-of-interest estimation device according to claim 1, wherein the attribute of the person is at least one of age and sexuality.

4. The degree-of-interest estimation device according to claim 3, wherein the first pulse correction unit performs a correction by multiplying the pulse of the person obtained by the pulse acquisition unit by a predetermined pulse correction coefficient by age and a predetermined pulse correction coefficient by sexuality according to at least one of the age and the sexuality, which are recognized by the attribute recognizer.

5. The degree-of-interest estimation device according to claim 1, further comprising an imaging unit configured to acquire the moving image by photographing the crowd stimulated by the object.

6. A degree-of-interest estimation method for estimating a degree-of-interest of a crowd with respect to an object, the degree-of-interest estimation method comprising:
inputting a moving image in which the crowd stimulated by the object is photographed;
recognizing existence of each person constituting the crowd based on the moving image;
obtaining a pulse of the person based on a luminance change of a skin of the person in the moving image;
recognizing an attribute of the person in the moving image;
correcting the pulse of the person so as to eliminate a difference in pulse depending on the attribute;
obtaining a statistical processing value of the pulse of the crowd by statistically processing the corrected pulse of the person; and
outputting a numerical index corresponding to the statistical processing value of the pulse of the crowd as the degree-of-interest.

7. A non-transitory computer-readable recording medium storing a program causing a computer to perform the degree-of-interest estimation method according to claim 6.

8. (canceled)

9. The degree-of-interest estimation device according to claim 2, wherein the attribute of the person is at least one of age and sexuality.

10. The degree-of-interest estimation device according to claim 9, wherein the first pulse correction unit performs a correction by multiplying the pulse of the person obtained by the pulse acquisition unit by a predetermined pulse correction coefficient by age and a predetermined pulse correction coefficient by sexuality according to at least one of the age and the sexuality, which are recognized by the attribute recognizer.

11. The degree-of-interest estimation device according to claim 2, further comprising an imaging unit configured to acquire the moving image by photographing the crowd stimulated by the object.

12. The degree-of-interest estimation device according to claim 3, further comprising an imaging unit configured to acquire the moving image by photographing the crowd stimulated by the object.

13. The degree-of-interest estimation device according to claim 4, further comprising an imaging unit configured to acquire the moving image by photographing the crowd stimulated by the object.

14. The degree-of-interest estimation device according to claim 9, further comprising an imaging unit configured to acquire the moving image by photographing the crowd stimulated by the object.

15. The degree-of-interest estimation device according to claim 10, further comprising an imaging unit configured to acquire the moving image by photographing the crowd stimulated by the object.
